(19) United States
(12) Patent Application Publication
(10) Pub. No.: US 2022/0104929 A1
(43) Pub. Date: Apr. 7, 2022

(54) BOVINE MOTION SENSOR TAG

(71) Applicant: AGTAG LIMITED, County Clare (IE)

(72) Inventor: Tim Cummins, County Clare (IE)

(21) Appl. No.: 17/429,465

(22) PCT Filed: Feb. 10, 2020
(86) PCT No.: PCT/EP2020/053365

§ 371 Date: Aug. 9, 2021

(30) Foreign Application Priority Data
Feb. 8, 2019 (GB) 1901826.6

(51) Int. Cl. A61D 17/00; A01K 11/00; G16H 40/63

(52) U.S. Cl. CPC A61D 17/008; G16H 40/63; A01K 11/006; A61D 17/002

(57) ABSTRACT

Aspects of the present invention relate to an apparatus and a method of determining when a cow may be in oestrus (in-heat), or when the cow is about to calve. The method comprises monitoring movement of the cow using a motion sensor or sensors attached to the cow. The method further comprises determining a mathematical function of the movement pattern of the cow based on the monitored movement of the cow over a period of time, and determining when the cow is in heat or about to calve by analysing and comparing the mathematical function to threshold values which are adjustable by a machine-learning self-adjusting algorithm.

### TECHNICAL FIELD

The present disclosure relates to a bovine motion sensor tag and in particular but not exclusively, to a calving detection system and to an oestrus or heat detection device. Aspects of the invention relate to a method of determining when a pregnant cow is in heat or about to calve, to a system of determining when a cow is in heat or about to calve, to a sensor tag and to a neck-mounted collar sensor system.

### BACKGROUND

Missing a cow-servicing by a few weeks is a major financial issue for farmers, because of losing a few weeks of milk production 9 months later. Losing a calf (and maybe also the cow) due to an unattended difficult birth is also a well-known animal welfare issue and a financial cost issue for farmers. For calving, regular monitoring of the cow is therefore important to avoid this, e.g. several times a day, an even hourly, round the clock as birth approaches. But this is not practical for many busy farmers. Cameras and CCTV monitoring systems relieve the burden slightly, but still require regular night waking and monitoring. Similarly the busy farmer cannot easily monitor cows in the field continuously to identify the narrow window of less than a day when the cow is in heat (oestrus) and ovulating.

Many technology solutions have sought to address these issues. These generally take advantage of known changes in behaviour of the animal in heat (e.g. more walking, restlessness butting, mounting, —DuPonte 2007), and more walking, pacing, lying down/getting-up as parturition approaches (Titler et al 2015).

Various sensor monitoring systems have become available for attaching to or inserting in the cow, for giving advance oestrus and pending-birth predictions. A selection of prior art systems are outlined briefly below.

Bolus rumen sensors (e.g. WO 2011/079338), have been used to detect temperature & movement changes in cows. However the alert for calving may issue 8 to 36 hours before birth, which may not be useful or practical for a busy farmer. Furthermore, many farmers are not comfortable with invasive sensors, which typically need a veterinarian to install.

Pedometers Firk et al (2003) used pedometers to monitor increased walking and movement patterns in 862 cows to detect oestrus. Valenza et al (2010) similarly used leg-mounted accelerometers to develop an ‘activity index’ predictor of oestrus. (Chebel et al 2013), used motion sensors to detect movement, pawing, and restlessness which increases in the first stage of labour (Wehrend et al 2006, Miedema et al 2011), and/or increased frequency of transition from lying to standing (Schuenemann et al 2011, and Titler et al 2015, who similarly proposes an ‘activity index’ for birth prediction). Titler (Ohio State Univ, 2015) uses a pedometer to predict calving, but the range of advance alert is 2 hours to 14 hours, which is not useful or practical for the busy farmer.

Temperature or light calving sensors inserted into the vagina near the cervix, e.g. U.S. Pat. No. 3,583,389. These detect a sudden change of temperature or light when expelled by the amniotic sac shortly before birth. However these typically require a skilled person or veterinarian to insert, which is expensive and not always practical. Many farmers hesitate to use these invasive devices, due to risk of infection, and possible distress to the animal and risks to the unborn foetus. Furthermore, the amniotic sac expulsion may be too late an indicator for the farmer to assist in the event of a difficult birth, since the foetus has already entered the birth canal.

Electro-mechanical sensors: e.g. FR2618051 (1987, Tilt-switch apparatus), FR2618051 (1987, Tilt switch), GB2257886 (1991 Tilt-Switch), U.S. Pat. No. 5,511,460 (1996 Tilt-Switch+metal harness). These calving sensors rely on monitoring the trait of the cow's tail raising for repeated and sustained periods when going into and through labour. An alarm is issued if the tail is raised horizontally, e.g. for 100 seconds (GB1579807), or for 4 to 12 minutes (EP0377343), or for 3 minutes (GB2257886). It is self-evident that while these may work some of the time on some animals, they will not work reliably across a broad range of animals where tail-raise times can vary widely. They are also bulky and prone to false alarm issuance due to sudden mechanical movements or shocks. Most of these systems have failed to gain any significant commercial market traction.

EP0705533 is an oestrus (‘in-heal’) detection system, comprising a mercury-in-glass tilt-sensor mounted on the cow's neck, and/or moving ball on electrodes, for detecting the cow's neck and head movements and eating behaviour, and changes of these indicative of the cow potentially being in oestrus. This has the same disadvantages of being prone to sudden mechanical movements or shocks. It has the additional limitation of data upload only when the cow comes into the milking parlour. This is clearly not suitable for oestrus detection in beef and suckler cows roaming in fields, where the farmer may not see them regularly, or where they do not come near the farm shed or houses regularly.

Accelerometer/solid-state oestrus/heat sensors: US 2010/0030036 describes accelerometer based collar and leg sensors for health and fertility/oestrus monitoring. JP2011/234668 describes accelerometer based leg and tail mounted sensors for oestrus/heat detection. Being solid-state, both of these disclosures are more reliable and less prone to mechanical shocks than electro-mechanical sensors. However, they require that the animal passes near a transponder, for example at milking time, for the bovine movement data to be received and analysed. This can create a time-lag of up to 12 hours, resulting in missed heat detection. For non-milking (suckler) outdoor animals, detection is not possible at all.

Accelerometer/solid-state calving/parturition sensors: JP2011/234668, WO2013/186232/EP3134478, and US2015/0230903 all disclose calving prediction by tail-mounted accelerometer sensors. However, these systems are large, heavy (>300 grams), and bulky thus requiring a ratchet clamp or a lot of duck-tape wrapping around the cows tail to achieve a secure mounting. This can be quite annoying to some cows and often the cows try to dislodge it, causing false alarms in many cases. Furthermore the heavy units and duck-tape or ratchet clamps may cause a sore and swollen tail if left in position for a few days. Tail amputations have been reported in some cases (A. Lind, Proceedings Animal Welfare Science, 2017). These units are bulky and heavy, at least partly, due to the large battery that is required to power the radio link or the GSM data transmissions. For example, up to 2 Watt peak power to get a reliable connection to remote GSM cell-towers. Thus some sensors alert the farmer to remove the sensor if it has been on the tail for more than 3 or 4 days. But this renders them impractical for the busy farmer who may not know the exact expected calving date. The 2W peak transmit power can drain the battery rapidly, particularly if the cow is in a remote area with poor coverage, or in a steel shed where GSM frequencies (1.8 GHz or higher) do not penetrate very well. This leads to false-negative missed calving alerts.

Many calving detection methods have been proposed, for example the pitch & roll equations as outlined in WO 2013/186235 to calculate the tail angle. While these work well for airplanes flying horizontally, they can be unreliable when mounted on a cow's tail. This is due to the ‘Gimbal lock’ effect as the tail moves from horizontal to vertical, causing data from one of the accelerometers to become unreliable and ‘noisy’ as it becomes parallel to gravity, resulting in occasional false positive alarms

WO 2017/211473 uses a simpler algorithm (tail raise by >10° for 2 to 30 seconds), with a ‘leakage accumulator’ to increment or decrement a “contraction counter” over a 30 to 50 minute rolling average time window. It relies on a 4 to 6-minute timing gap between contractions to distinguish whether or not birth is imminent. Once again this may work for some cows, but not all, resulting in occasional ‘false negatives’, e.g. where the cow was tired and just ‘took a break’ for a bit longer between contractions, and the farmer may miss the birth event; or false positives where the increased tail activity may be due to feeding, defecating, or other nearby animal activity.

WO 2017/211473 also introduces magnetometer and gyro sensors, in ‘sensor fusion’ as ‘low-pass filtering’. However, a gyro is a high-pass filter—it detects and emits a signal for sudden angular movements, but has no output when stationary, i.e. no low-frequency component. Thus this method of measuring contractions is not satisfactory.

Missed heats, missed calvings, sore tails, sensor dislodging, false-negatives and false-positive alarms are therefore an ongoing issue with all the above sensors and methods. It is an aim of the present invention to address one or more of the disadvantages associated with the prior art.

### SUMMARY OF THE INVENTION

This invention disclosure describes a lightweight sensor tag for predicting when a cow is in heat and ready for insemination; and 9 months later predicting and alerting 1 to 3 hours in advance of when she is about to give birth to a resulting calf. It integrates 9-axis motion sensors with a 32-bit microcontroller, which implements an advanced machine-learning algorithm to adapt in real-time to each individual cows movement patterns. For heat detection, it can be slotted into the cow's ear-tag, or mounted in a neck belt, or in a foot-strap as a pedometer. For calving detection, it can be stuck on the cow's tail with an adhesive, just like sticking on a paper ‘heat tag’ with Kamar adhesive which farmers are familiar with. Weighing only 10 gm, it is light enough to be almost imperceptible to the cow. Or alternatively it can be attached to the tail with a medical-grade crepe elastane bandage with Velcro, which is quite comfortable and imperceptible for the cow. This eliminates the well-known issues of heavier sensors which the cow tries to knock off due to annoyance, or which cause sores and swelling of her tail due to the tight clamping required to hold them in position.

It is completely sealed, to IP67 protection level, which is important for a tail-mounted sensor unit in the vicinity of urine and faeces. It is self-powered by a thin internal battery which is wireless rechargeable. It accurately tracks the cows walking and lying movement patterns, and identifies oestrus by known behaviour changes. Similarly, when mounted on the tail, it additionally tracks tail movements and angles, as well as lying, walking, and moving patterns, to create an activity index to identify the stages of labour, and additionally a ‘probability index’ to reliably issue the birth alert while minimising false positives and false negatives.

It has LoRa low-power kilometer-range 433 MHz/868 MHz wireless communication to a base unit. This relays the sensor status or alerts to the farmer via GSM to his phone, or via his local WiFi to his PC or TV. The sub-GHz LoRa tag frequency can travel more easily through walls and sheds, and for distances of a few kM, for example 1, 2, or 3 km, eliminating the ‘loss-of-signal’ false-negative problems of other line-of-sight GHz wireless sensors, and ‘blind-spot’ coverage issues of GSM sensors.

According to an aspect of the present invention there is provided a method of determining when a pregnant cow is about to calve, the method comprising: monitoring movement of the cow using a motion sensor attached to the cow; determining a movement pattern of the cow based on the monitored movement of the cow over a period of time; and determining when the cow is about to calve by comparing the determined movement pattern with a stored movement pattern that is representative of such a cow calving. The method may comprise determining a mathematical function of a movement pattern of the cow based on the monitored movement of the cow over a period of time wherein the mathematical function is a calving activity index; and determining that the cow is about to calve when the calving activity index exceeds a threshold value; wherein the threshold value is adjusted up or down by a probability index indicative of the probability the cow has started labour.

Comparing the determined movement pattern with a stored movement pattern beneficially improves the accuracy of determining when the cow is about to calve. The stored pattern may be a generic movement pattern associated with a cow calving or the stored pattern may be adapted to the cow that is being monitored. For example, the stored movement pattern may be a pattern that the cow followed in a previous calving year or the stored pattern may be a known movement pattern for a species of the cow.

In one embodiment the probability index may be determined by dividing the number of steps the cow has taken within a time period by the time the cow spent standing in said time period. The threshold value of the calving activity index may be between 5 and 50. For example, the threshold value may be 10 or 20. The threshold value may be adjusted in dependence on the breed of the cow or the birthing history of the cow that the motion sensor is attached to.

In another embodiment the calving activity index may be calculated by multiplying the probability index by: (lying bouts/hr)²*\sqrt{no.of.tail raises/hr}. In one embodiment the method may comprise scanning an electronic ID tag of the cow and adjusting the threshold value in dependence on the scanned electronic ID tag. In another embodiment the method may comprise scanning a non-electronic ID tag of the cow and adjusting the threshold in dependence on the scanned electronic ID tag.

In an embodiment the method may comprise adjusting a duty cycle of the motion sensor in dependence on the movement pattern of the cow. This is beneficial as the duty cycle may be increased when the cow is active thereby improving the resolution of data gathered when the cow is active. Furthermore, the duty cycle may be decreased when the cow is inactive and thus not moving. This is beneficial as it reduces power consumption of the battery thereby allowing the battery to be smaller. Adjusting the duty cycle may vary a sample rate of movement data being generated by the motion sensor. The movement data may be, for example, acceleration data generated by an accelerometer.

In an embodiment comparing the determined movement pattern with the stored movement may comprise selecting the stored movement pattern from a plurality of stored movement patterns.

In another embodiment the stored movement pattern may be selected in dependence on the cow the motion sensor is attached to. This is advantageous as each cow may calve differently depending on factors such as the breed of the cow, the calving history of the cow, the age of the cow and as such this improves the accuracy of determining when the cow is calving based by tailoring the response to such a cow.

In one embodiment the stored movement pattern may be selected in dependence on the breed of the cow the motion sensor is attached to. In another embodiment the stored movement pattern may be selected in dependence on a calving history of the cow.

In an embodiment determining when the cow is about to calve may comprise comparing the determined movement pattern with an expected due date. This is beneficial as it allows the determining step to include an expected due date. For example, if the cow is expected to calve in two weeks' time, then the chance that a possible pattern match, as determined in the comparing step, is a false positive. As such, the threshold for determining a pattern match in the comparing step may be more stringent when the due date is some time away. To the contrary, if the cow is expected to calve within the next 24 hours then the comparing step may reduce the threshold of the pattern match as the chance that the cow is calving is increased.

In another embodiment the method may comprise generating an alert that the cow is about to calve. This is beneficial as the alert may be sent to notify a person, such as the farmer, that the cow is calving and that the cow may require assistance. The alert may include information indicating if the cow is experiencing difficulty in the calving experience or the alert may indicate that the calving process is proceeding as planned. This is beneficial as it indicates a level of urgency of assistance that the cow may require during calving.

In one embodiment determining a movement pattern of the cow may comprise filtering the monitored movement. The filtering step may include the use of a Kalman filter or a Bayes filter. Filtering the data in this manner reduces the likelihood of noise or sudden unusual changes in the movement signal causing a false-positive or a false-negative.

In another embodiment the method may further comprise: monitoring the movement of the cow before the cow is about to calve; determining a movement pattern of the cow before the cow begins calving; determining when the cow is about to calve by comparing the determined movement pattern of the cow based on the monitored movement of the cow with the determined movement pattern of the cow before the cow begins calving. This is beneficial as the typical movements of the cow may be monitored such that a movement pattern is established. If over a period of time the monitored movement pattern diverges from the known, typical movement pattern then it may be determined that the cow is calving.

In an embodiment the method may comprise scanning an electronic ID tag of the cow and selecting the stored movement pattern in dependence on the scanned electronic ID tag. Scanning ID tag of the cow allows a movement pattern to be selected that is appropriate to the pregnant cow. For example, the selected movement pattern may be a movement pattern that was recorded in the previous year, a movement pattern typical to the breed of the cow or a movement pattern of a relative to the cow.

In one embodiment the method may further comprise determining the orientation of the sensor tag relative to the cow. For example, the Y gravity vector while cow is standing is −1 g, indicating the Y axis is pointing down to the ground. Whereas when the X gravity vector is +1 g, indicating to the processor that X is pointing up, therefore Y is pointing horizontally left. The orientation of the sensor tag may be determined relative to the cow at least partially in dependence on the monitored movement of the cow. The method may further comprise determining a correction factor to be applied to the monitored movements of the cow based on the determined orientation of the sensor tag relative to the cow, for example ‘swapping’ the X, Y, and Z axes reference coordinate system, based on the tag orientation or rotation. The method may further comprise applying the determined correction factor to the monitored movement data.

According to another aspect of the present invention there is provided a system for determining when a pregnant cow is about to calve, the system comprising: a movement sensor attached to the cow being configured to monitor movement of the cow; a memory configured to store a movement pattern that is representative of such a cow calving; and a controller configured to determine a movement pattern of the cow based on the monitored movement of the cow over a period of time; wherein the controller is further configured to determine when the cow is about to calve by comparing the determined movement pattern with a stored movement pattern that is representative of such a cow calving. The controller may be configured to calculate a mathematical function of a movement pattern of the cow based on the monitored movement of the cow over a period of time wherein the mathematical function is a calving activity index and wherein the controller is further configured to determine that the cow is about to calve when the calving activity index exceeds a threshold value and wherein the threshold value is adjusted up or down by a probability index indicative of the probability the cow has started labour.

In an embodiment the system may comprise a communication module. In another embodiment the communication module may be configured to provide a notification or alert indicative of the predicted calving time for the cow to a mobile communication device. The communication module may comprise a long-range communication module configured to transmit the notification or alert to the mobile communication device. The communication module may comprise a near-field-communication module configured to communicate with an electronic ID tag of the cow.

In another embodiment the controller may be configured to select a stored movement pattern based on data received from the electronic ID tag.

In one embodiment the system may comprise a filter module configured to filter noise in the monitored movement of the cow.

In another embodiment the system may comprise a collar or neck mounted sensor configured to receive data indicative of the monitored movement from the movement sensor and wherein the collar is configured to transmit the received data to a mobile communications device. This is beneficial as the movement sensor is only required to transmit data from the cow's tail to neck thereby reducing the power requirements of the transmitter in the sensor tag which in turn reduces the overall size of the movement sensor.

The collar may be configured to communicate with one or more sensor tags fitted to other cows. In this sense the collar may act as a base station for the other sensors on the cow, or for the entire herd.

In an embodiment the motion sensor is secured to the cow's tail. In another embodiment the system may comprise a further motion sensor secured to one of: a leg, neck and ear of the cow.

In an embodiment the controller may be configured to determine the orientation of the movement sensor relative to the cow in dependence on the movement of the cow. This is beneficial as the sensor tag may be mounted to the cow in any orientation and then the controller may determine the orientation based on the monitored movement of the cow. Furthermore, the sensor tag may move relative to the cow during use and as such the controller may update the position of the sensor tag relative to the cow to ensure the monitored movement is correct. For example, a correction factor may be determined by the controller and applied to the monitored movement data.

According to another aspect of the present invention there is provided a sensor tag for use in any of the aforementioned embodiments and aspects of the present invention. According to a yet further aspect of the present invention there is provided a collar for use in any of the aforementioned embodiments and aspects of the present invention.

According to an aspect of the present invention there is provided a self-powered motion sensor tag that is attachable to the tail of a cow to determine when the cow is about to calve by reference to a mathematical function of the cow's movements, wherein the tag is configured to emit a wireless signal that is indicative of the cow calving and comprises: an adhesive for attaching the tag to the tail of the cow; and
a housing containing: at least one three-axis motion sensor for determining the cow's movements; a controller that is responsive to the motion sensor to generate said mathematical function and said signal, wherein the mathematical function is a calving activity index indicative of the probability the cow has started labour and a wireless communication module and an antenna for emitting said signal wirelessly, wherein the tag weighs less than 20 grams and is less than 30 mm diameter.

The sensor tag may be configured to emit the wireless signal to a first mobile communication device, such as a mobile phone, acting as a gateway unit. The first mobile communication device may be configured to receive data from the sensor tag and to send a notification to a second mobile communication device, such as a mobile phone belonging to the farmer.

In one embodiment the sensor tag may comprise a strap configured to secure the sensor tag to the tail of the cow. This is beneficial as the strap may be fabric and beneficially the strap does not cause soreness or irritation to the cow's tail when it is secured. The strap may comprise a pocket configured to receive the sensor tag. Beneficially, locating the sensor tag within a pocket reduces the likelihood that the sensor tag may get caught and dislodged from the tail of the cow.

The strap may comprise an adjustable attachment element such as a Velcro element. This provides the advantage of easily being able to secure the strap to the tail of the cow. Furthermore, the Velcro element allows the tightness of the strap on the cow's tail to be adjusted to reduce the chance that the strap will cause soreness or irritation to the cow's tail.

In an embodiment the sensor tag may weigh less than 20 g. In another embodiment the tag may be less than 10 grams and have a diameter of 25 mm or less. This is beneficial as a lightweight sensor tag may be easily secured to the tail of the cow thereby reducing the annoyance or irritation to the cow. The sensor tag may comprise a LoRa communication module to transmit data. This is beneficial as LoRa is low power and long range and as such the sensor tag does not require a large battery thereby minimising the weight of the sensor tag.

The tag may be attached to the outer hairs of the tail. The tag may be secured to the tail hairs by a wrap-around breathable fabric. The fabric may comprise a Velcro fastener. The sensor tag may only be removable by cutting or pulling out the hairs or be moulting of the hairs.

In another embodiment the calving activity index may be calculated by a processor in the controller as a calving probability index multiplied by (lying bouts/hr)²*\sqrt{no.of.tail raises/hr}. The calving probability index may be calculated by dividing the number of steps the cow has taken within a time period by the time the cow spent standing in said time period.

In one embodiment the calving activity index may be compared to a threshold value. In another embodiment the controller may comprise a Bayes filter and/or a Kalman filter. The Bayes and/or the Kalman filter may be configured to filter movement data generated by the movement sensor and to vary the calving activity index up or down in dependence on the filtered movement data.

The tag may have a range of transmission of the wireless signal of 50 m or less. In another embodiment the range may be 20 m or less.

According to a further aspect of the present invention there is provided a method of securing a sensor tag to the tail of a cow using a strap, the method comprising: positioning the strap on the tail of a cow; securing the strap to the tail of the cow with an adhesive; and fastening the strap to the tail of the cow.

Securing the sensor tag to the tail of the cow with an adhesive is beneficial as it prevents the strap and sensor tag sliding down the tail of the cow over a period of time. Further the adhesive prevents the strap and sensor tag rotating on the tail. This is beneficial as it reduces the chance that the sensor tag will move relative to the tail or become dislodged.

In an embodiment the method may comprise locating the sensor tag within a pocket of the strap. This is beneficial as the pocket protects the sensor tag and reduces the chance that it will get caught and potentially dislodged from the tail of the cow.

According to another aspect of the present invention there is provided a sensor tag for determining when a pregnant cow is about to calve, the sensor tag comprising: a movement sensor configured to monitor movement of the cow; a memory configured to store a movement profile of the cow derived from movement of the cow monitored by the movement sensor; a controller configured to determine a change in the monitored movement of the cow compared to the stored movement profile, which change is indicative of the cow calving; and a sub-gigahertz communication module that is responsive to the controller determining said change in the movement of the cow to transmit a cow calving notification to a mobile communication device.

Beneficially, the sub-gigahertz communication module has low power consumption and as such the battery used to power the sensor tag may be small and lightweight. This allows the sensor tag to be secured to the tail of the cow more easily and reduces the annoyance and soreness that the cow may experience. Furthermore, sub-giga hertz communication, such as long range communication, may transmit data in excess of 1000 m through obstacles which reduces the chance of the notification not being received by the mobile communication device.

According to a further aspect of the present invention there is provided a self-powered motion sensor tag that is attachable to the tail of a cow to determine when the cow is about to calve by reference to a movement pattern of the tail, wherein the tag is configured to emit a wireless signal that is indicative of the cow calving and comprises: a bovine adhesive for attaching the tag to the tail of the cow; and a housing containing: at least one three-axis motion sensor for determining the movement pattern; a processor that is responsive to the motion sensor to generate said signal, and a wireless communication module and an antenna for emitting said signal wirelessly.

In an embodiment the tag may comprise a strap that is arranged to be wrapped around the tail. In another embodiment the adhesive may be applied to the strap. In one embodiment the strap may comprise a pocket for receiving the housing. The motion sensor tag may weigh less than 20 grams. In another embodiment the tag may further comprise a surrounding bandage of conformable stretch fabric.

According to a yet further aspect of the present invention there is provided a method of determining when a pregnant cow is about to calve, the method comprising: monitoring movement of the cow using a motion sensor attached to the cow; varying a sample rate of movement data generated by the motion sensor in dependence on the monitored movement; and determining when the cow is about to calve by comparing the monitored movement with a movement pattern.

In an embodiment the sample rate may be reduced when the monitored movement of the cow indicates that the cow is inactive. The cow may be considered to be inactive when it is lying down or when it is stood still for a prolonged period. For example, one minute or longer.

In another embodiment the sample rate may be increased when the monitored movement of the cow indicates that the cow is active. For example, if the cow is walking, lying, or moving her tail.

According to a yet further aspect of the present invention there is provided a system for determining when a pregnant cow is about to calve, the system comprising: a motion sensor attached to the cow being configured to monitor movement of the cow; a collar sensor system attached to the cow's neck being configured to receive the monitored movement of the cow from the motion sensor; and wherein the collar is configured to transmit a signal indicative of the received monitored movement to a remote communication device.

In one embodiment the system may comprise two or more motion sensors attached to respective cows being configured to transmit monitored movement of the respective cows to the collar. The collar may comprise a GSM and/or Wi-Fi module configured to transmit the signal to the remote communication device. The collar may further comprise a further motion sensor configured to monitor movement of the cow.

The motion sensor may be configured to adjust the rate at which data indicative of the monitored movement of the cow is transmitted to the collar in dependence on the movement of the cow. For example, the sensor may increase the rate at which data is sent to the collar when the cow is active or moving. Similarly, the sensor may decrease the rate at which data is sent to the collar when the cow is inactive or not moving.

According to a yet further aspect of the present invention there is provided a motion sensor tag configured to configured to be attached to the tail of a cow to determine when the cow is in oestrus by reference to a mathematical function indicative of the cow's movements, wherein the tag is configured to emit a wireless signal that is indicative of the cow in oestrus and comprises: an adhesive for attaching the tag to the tail of a cow; and a housing containing: at least one three-axis motion sensor for determining the cow's movement; a capacitive proximity sensor configured to detect another animal mounting the cow; a controller that is responsive to the motion sensor to generate said mathematical function and being configured to combine the mathematical function with proximity sensor data to generate said signal; and a wireless communication module and an antenna for emitting said signal wirelessly.

In an embodiment the tag may weigh less than 20 grams. In another embodiment the tag may weigh less than 10 grams.

In a further embodiment the capacitive proximity sensor may comprise a charge-balancing second-order sigma delta converter. The charge balancing second-order sigma-delta convertor may resolve 1 femtoFarad to 10 femtoFarad capacitance variation for another animal in 15 to 150 mm proximity of the tag.

In one embodiment the tag may be configured to be attached to the outer hairs of the tail. For example, with an adhesive. The tag may be removed by cutting, pulling out the tail hairs or by moulting of the hairs.

In a further embodiment the mathematical function may be calculated from the cow's movement and the cow's movement may be indicative of the cow's walking, standing and lying movements. In one embodiment the oestrus may be determined by the cow remaining still for a period of time while the cow is mounted by another animal. The controller may be configured to determine that the cow is in oestrus when the motion sensor detects a period of inactivity and the proximity sensor detects that the cow has been mounted by another animal. The period of inactivity may be a period where the cow is standing but not taking any steps. For example, the controller may determine that the cow is standing still based on the gathered motion sensor data.

In an embodiment the controller may comprise a recursive filter configured to combine motion sensor data and capacitive proximity sensor data to determine oestrus.

According to a further aspect of the present invention there is provided a system for determining when a cow is in oestrus, the system comprising: a capacitive-proximity sensor tag configured to be mounted to the tail of the cow and further being configured to determine another animal mounts the cow, in use; and a collar configured to be placed around the cow's neck wherein the collar comprises: at least one three-axis motion sensor for determining the cow's movements; a wireless communication module configured to communicate with the capacitive-proximity sensor; a controller configured to generate a mathematical function indicative of the cow's movements and further being configured to determine that the cow is in oestrus in dependence on the mathematical function and data from the capacitive-proximity sensor; wherein the wireless communication module is configured to emit a signal to a remote communication device when the controller determines that the cow is in oestrus.

In one embodiment the capacitive proximity sensor may comprise a charge-balancing second-order sigma-delta converter. The capacitive sigma-delta converter may resolve 1 femtorFarad to 10 femtoFarad capacitance variation for another animal 15 mm to 150 mm proximity of the tag. The sensor tag may weigh less than 20 grams, for example 10 grams.

In another embodiment the mathematical function may be calculated from the cow's movement and wherein the cow's movement is indicative of one or more of: the cow's walking, standing and lying movements. The mathematical function may additionally be calculated in dependence on the cow's neck and head movements. The oestrus may be determined by the cow remaining still while mounted by another animal.

In one embodiment the controller may comprise a recursive filter configured to combine to combine motion sensor data and capacitive proximity sensor data to determine when the cow is in oestrus. In another embodiment the collar may additionally comprise a GPS location sensor configured to monitor the position of the cow. The position of the cow may be used to determine when the cow has been mounted. For example, if the cow is shown as being at a feeding area then it is likely that the proximity sensor will indicate that the cow is in close proximity with other animals. However, if the cow is shown to be at a feeding area it may be inferred that the proximity is a result of feeding near other animals opposed to being mounted.

In an embodiment the signal emitted by the communication module may be indicative of the position of the cow and an ID number of the cow. In another embodiment the system may comprise a head movement sensor configured to be mounted to the cow's ear to monitor movements of the cow. For example, the ear sensor may monitor movements of the cow's head that are indicative of walking, grazing, lying or standing. In another embodiment the system may comprise a leg movement sensor configured to be mounted to the cow's leg to monitor movements of the cow. For example, the sensor may monitor movements of the cow's leg indicative of walking, grazing, lying or standing. The head movement sensor and/or the leg movement sensor may be configured to communicate with the collar.

The controller may be configured to determine when the cow is in oestrus by comparing the cow's movement and mounting data with average movement data for a herd of cows.

### BRIEF DESCRIPTION OF THE DRAWINGS

One or more embodiments of the invention will now be described, by way of example only, with reference to the accompanying drawings, in which:

FIG. 1 is a schematic diagram of a cow fitted with sensor tags according to embodiments of the invention;

FIG. 2 is a schematic diagram of the sensor tag of FIG. 1;

FIG. 3 is a hardware block diagram of the sensing tag of FIG. 1;

FIG. 4 is a perspective view of a sensor tag suitable for use with embodiments of the invention;

FIG. 5 is an exploded perspective view of the sensor tag of FIG. 1;

FIG. 6 is a view of a sensor tag suitable for use with embodiments of the invention;

FIG. 7 is a perspective view of the sensor tag of FIG. 1 secured to the tail of a cow;

FIG. 8a is a perspective view of the sensor tag of FIG. 6 wrapped with fabric material;

FIG. 8b is a perspective view of the sensor tag of FIG. 6 stuck to the outer tail hairs of the cow;

FIG. 9 is a perspective view of a cow ID ear tag fitted with an embodiment of the sensor tag;

FIG. 10 is view of the sensor tag of FIG. 9 with the outer casing removed;

FIG. 11 is a hardware diagram of a sensor tag according to an embodiment of the invention;

FIG. 12 is a schematic of the accelerometer in the sensor tag of FIG. 1 labelled with X, Y and Z axis;

FIG. 13 is a detailed view of a time period of the accelerometer data of a cow's movements;

FIG. 14 is a graph of the data of FIG. 13 after being filtered by a moving-average filter;

FIG. 15 is a graph showing movement data when the cow's tail swishes from side to side;

FIG. 16 is a block diagram of a Kalman filter; and

FIGS. 17 (a) to (f) are graphs of the sensor tag data and calculations for a Holstein Friesian cow over five days, with calving occurring on day four.

### DETAILED DESCRIPTION

In general terms embodiments of the invention relate to a sensor device or tag configured to predict when a cow is in heat and ready for insemination. The sensor tag is also configured to determine when the cow is about to give birth to a calf and to provide a notification to the farmer of the approximate two hours and one hour before the time of calving. For example, a first notification may be sent approximately two hours prior to calving and a second notification may be sent approximately one hour prior to calving.

The sensor device comprises nine-axis motion sensors and a control module to monitor the movements of the cow to determine firstly when the cow is in heat and also when the cow is calving at the end of the pregnancy. The control module comprises a mathematical calculation and an artificial intelligence machine-learning algorithm to adapt in real-time to the movement patterns of each individual cow. This is beneficial as during heat or approaching parturition each cow may have a unique movement pattern. The algorithm may consider factors such as the cow's breed, number of previous calves, age and calving history to adapt parameters of the calving algorithm to that cow. It may similarly adjust the heat detection algorithm based on her breed, previous insemination history, number of mountings (assisted by the mounting proximity sensor), walking, pacing and eating behaviour, and indoor or outdoor housing conditions, which also affects her movements. This reduces the number of false positives and negatives that the farmer may receive about the cow, and improves the accuracy of the sensor alerts.

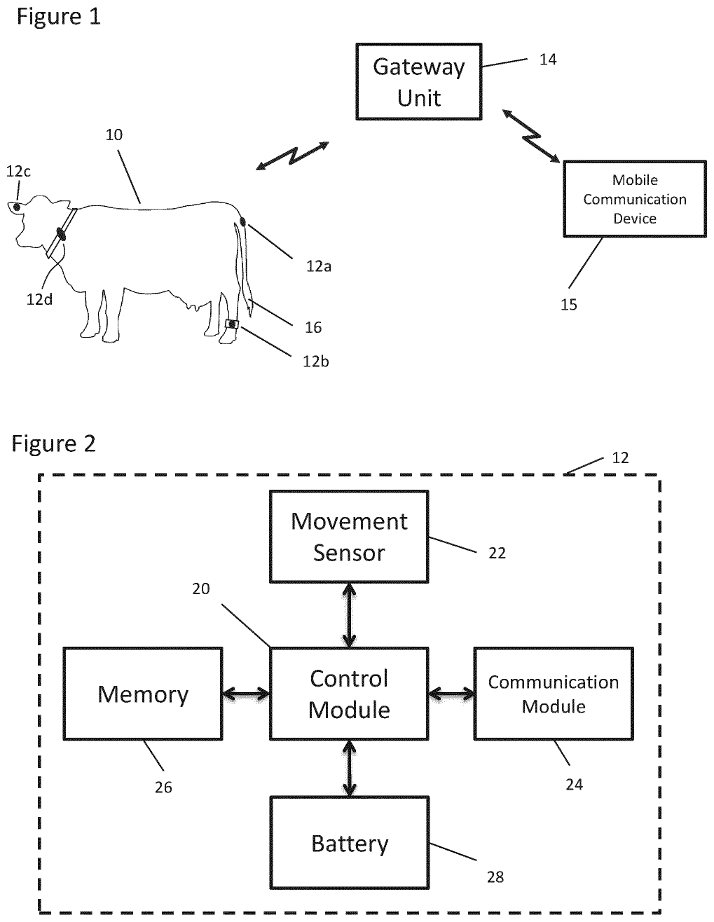

To place embodiments of the invention in a suitable context reference will firstly be made to FIG. 1 which shows a sensor device or tag 12a fitted to the tail 16 of a cow 10. The sensor tag 12 is configured to monitor the movements of the cow and the cow's tail 16 and to wirelessly transmit data indicative of the movements to the control or gateway unit 14. The movements of the tail sensor 12a are relevant to predicting when the cow 10 is about to calve, and the gateway unit 14 may provide notifications or alerts to a farmer to indicate when the cow is about to calve. The gateway unit 14 may communicate with a plurality of tags 12 fitted to cows, for example an entire herd, where each cow is fitted with a separate sensor tag 12a.

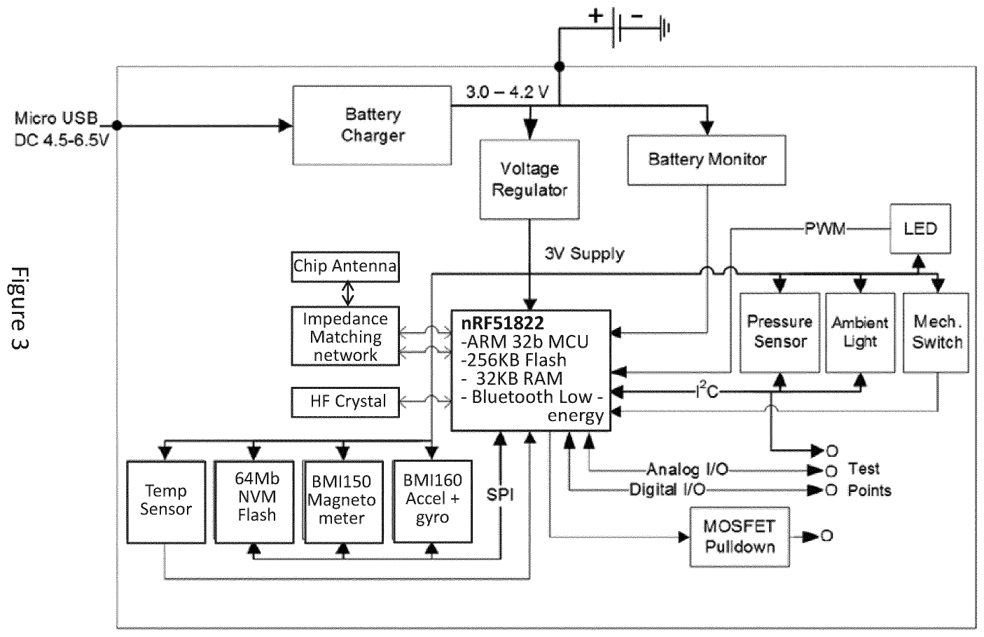

FIGS. 2 and 3 show a schematic of the sensor tag 12. In a broad sense as shown in FIG. 2, the sensor tag 12 comprises a control module 20, a memory unit 26, a wireless communication module 24, a battery 28 to power the tag 12, and a movement sensor 22 such as, for example, one or more of a three-axis accelerometer, three-axis gyroscope or three-axis magnetometer. The movement sensor 22 monitors the movements of the cow's tail 16 and transmits a signal indicative of the movements of the tail 16 to the control module 20. The data collected by the movement sensor 22 is stored in the memory unit 26 and may be communicated to the gateway unit 14 by the communication module 24. The movement data collected by the movement sensor 22 is indicative of movement of the cow's tail 16 in three dimensions as well as the movements and number of steps the cow takes, the length of time the cow is lying down or standing up, the number of times she lies down and gets up (lying bouts per hours), and also the side the cow is lying on when the cow is lying down. Alternatively, the tag 12a may store all the data in its memory 26 for processing and implementing of the machine-learning algorithm and mathematical calculations by the tag's processor, and conserve battery by only wirelessly transmitting short status bytes, at a very low duty-cycle, every 5 or 10 seconds (battery level, normal operating status, fault-detect, etc).

In an embodiment, as illustrated in FIG. 3 the controller or microcontroller unit (MCU) is an ARM 32b low-power processor, the communication module 24 is a Bluetooth low-energy (BLE) wireless communication device, the 3-axis Magnetometer is a Bosch BM1155, and a Bosch BM1160 provides the 3-axis accelerometers and 3-axis gyros. A 3-colour RGB LED is provided for indicating various communication and status conditions, and a button is provided for ‘hard reset’ and other functions, depending on duration pressed.

The Bluetooth low-energy (BLE) wireless communication device has a compact PCB-mounted chip antenna, with a range of approximately 50 m when a direct line of sight is available or about 20 m if there are obstacles or obstructions blocking the direct line of sight. Beneficially the controller, for example the ARM MCU, is a very low-power processor (˜10 mW during processing), and the (BLE) Bluetooth low-energy wireless communication module also uses very little power (˜20 mW) from the battery 28 at low data rates. Due to the relatively slow movements of the cow, the tag can spend 99% of its time in sleep mode (average current ˜2 uA), waking up typically for a few milli-seconds processing every 1 or 2 seconds, implementing the machine learning algorithm and mathematical calculations. And if it determines the cow is lying down, it can slow the sensor sampling rates even further, to less than one sample per second for example.

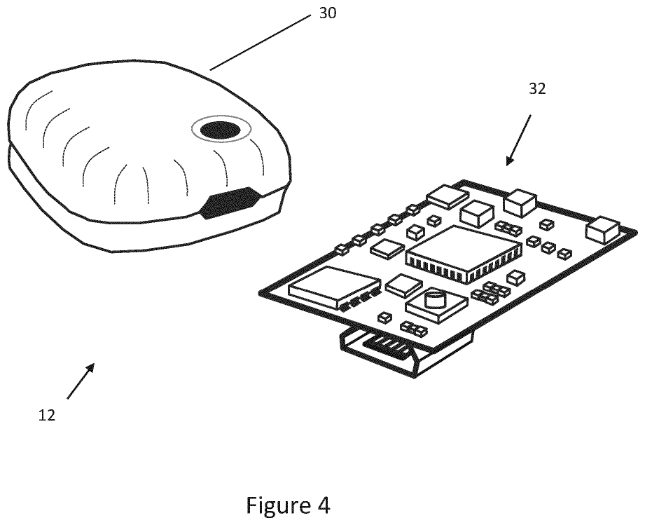

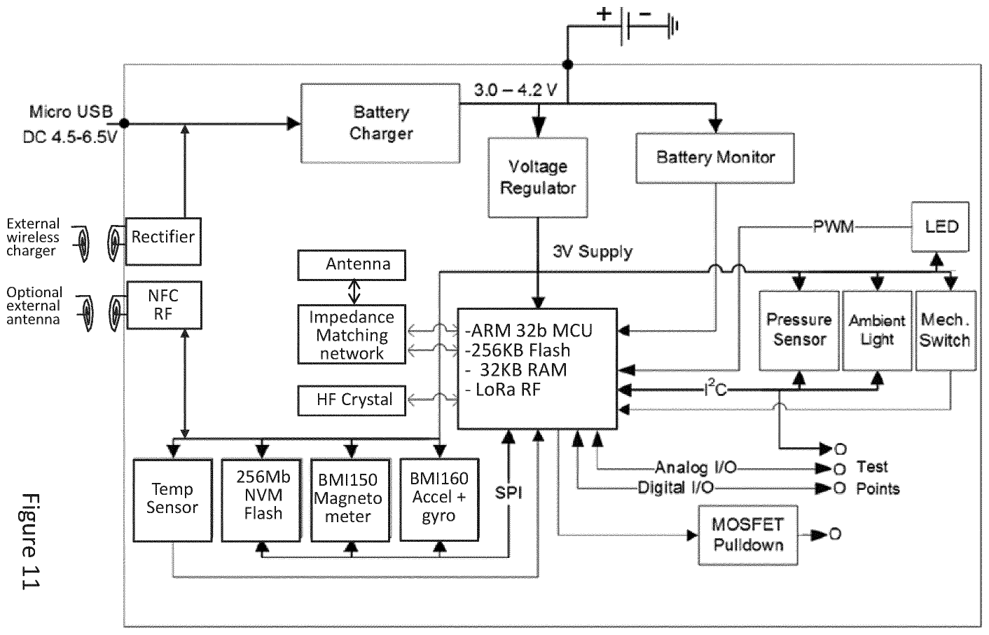

As such the battery life of the device 12 is prolonged significantly by this very low power processor and wireless duty-cycling. This is desirable as the farmer may fit the device 12 to the tail 16 of a cow many days or weeks prior to the cow commencing calving. The algorithm then ‘learns’ the cow's normal movements. This increases the detection accuracy when her movements change, during heat or onset of calving. And the tag can stay on the cows tail after calving for many weeks, to detect the onset of the cow's next oestrus and heat cycle. Furthermore, the low power consumption of the controller and communication module beneficially reduces the size of battery 28 required to power the sensor tag 12 thereby reducing the overall size and weight of the tag 12a, to 9 grams (FIG. 4), or to 7 grams and 25 mm diameter (FIG. 10), or to 5 grams and 20 mm diameter.

In this Bluetooth (BLE) embodiment of the device for indoor calving, the tag 12 may communicate directly with the gateway unit which may be a nearby communication device such as a permanently-powered mobile phone or laptop or base-station with GSM or WiFi. The gateway then relays the data or alert to the farmer's mobile communication device or phone, to provide a notification of a potential cow calving, or to a cloud server and database, for further storage, processing, or analysis. While the reduced range of 20 m seems counter intuitive and opposite of all prior art, this single-chip Bluetooth BLE processor is in fact key to achieving the tiny dimensions and lightweight, for example less than 10 g weight. This is key to solving the tail-swelling and welfare issues of the prior-art strap/ratchet/clamp/duck-tape bulky heavy sensors.

As shown in FIG. 1, the cow 10 may be fitted with a collar 12d. The collar 12d may act as a movement sensor and/or a gateway unit 14 such that it receives movement data from the tag 12a secured to the tail 16 of the cow 10 and, when it is determined the cow 10 is about to calve, transmit a notification to the mobile communication device 15 to alert the farmer that the cow 10 is going to calve. This is beneficial as the sensor tag 12a only has to transmit data over a short distance thereby reducing the battery and weight requirements of the tag 12a secured to the tail 16 of the cow 10. The collar 12d, when acting as the gateway unit 14 may comprise a battery and a communication module configured to transmit notifications to the mobile communication device, for example via GSM or Wi-Fi. The collar when acting as a movement sensor may additionally monitor the cows head, neck, walking, lying, and movement patterns, to facilitate oestrus detection.

When the system is used with a herd of cows a single cow 10 may be fitted with a collar 12d that acts as a gateway unit 14 for multiple cows within the herd. This is beneficial as a single collar 12d located on a cow can act as a gateway unit 14 for the entire herd fitted with sensor tags 12a. The large battery required to power a gateway unit 14 may easily be suspended around the neck of the cow 10 without causing discomfort or pain to the cow 10. This in turn, minimises the weight of the tag 12d secured to the tail 16 of each cow 10.

In another embodiment, suitable for outdoor cows, the communication module 24 is a long range (LoRa) wireless data communication module operating at a sub-gigahertz radio frequency, for example Semtech SX1261 transceiver operating at 433 MHz or 868 MHz. LoRa wireless communication is advantageous as it has a low power consumption (12 mW Rx, 25 mW Tx) while enabling the transfer of data over a longer range than the Bluetooth low-energy wireless communication device. The sub-gigahertz tag frequency can travel more easily through walls and sheds, the like of which may be found on farms, and around obstacles and hills for a distance of over 3 km, thereby eliminating “loss-of-signal” false-negative problems of other line-of-sight gigahertz wireless sensors. This is particularly advantageous for heat detection of suckler cows that are outdoors for many weeks or months, and for calving detection where the cow may be hidden from sight in a remote or secluded spot.

When cows are calving they will often remove themselves from the herd and rest in a remote or secluded spot. These spots are often behind a wall, in a ditch or hollow where the cow is out of sight and the signal from the device 12 is inhibited by surrounding obstacles. In this situation the LoRa wireless communication module advantageously maintains communication with the gateway unit 14 thereby ensuring that the farmer receives a notification of calving even when the cow is in a remote location and potentially hidden from sight.

In this embodiment the wireless communication module 24 may communicate with a gateway unit 14 as shown in FIG. 1. The gateway unit 14 is powered by mains electricity and is equipped with GSM and/or WiFi transceivers for onward transmission of data and birth alerts to a mobile communication device 15 such as a PC, phone or a cloud server and database. Alternatively, the communication module 24 may communicate with a collar 12d acting as the gateway unit 14. The collar 12d may be fitted to the cow 10 that is about to give birth or it may be fitted to another cow 10 within the herd.

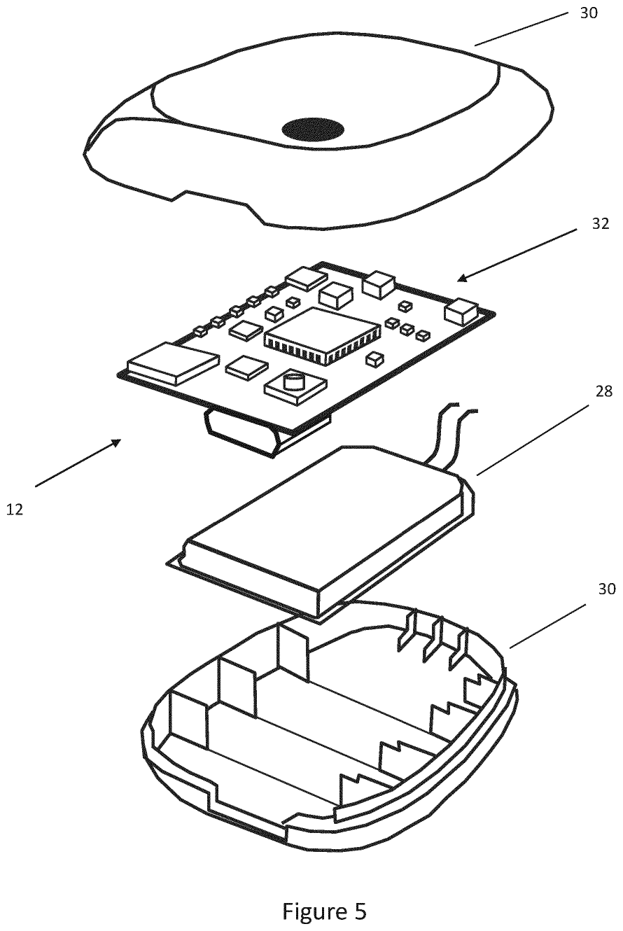

FIGS. 4 and 5 show a sensor device 12 suitable for use with embodiments of the invention. The device 12 comprises an outer casing 30 and a PCB 32. For illustrative purposes the PCB 32 in FIG. 4 is shown outside the casing, however, in use the outer casing 30 encompasses and seals the PCB. The device 12 may be completely sealed, to IP67 protection level, which is important for a tail-mounted sensor unit in the vicinity of urine and faeces.

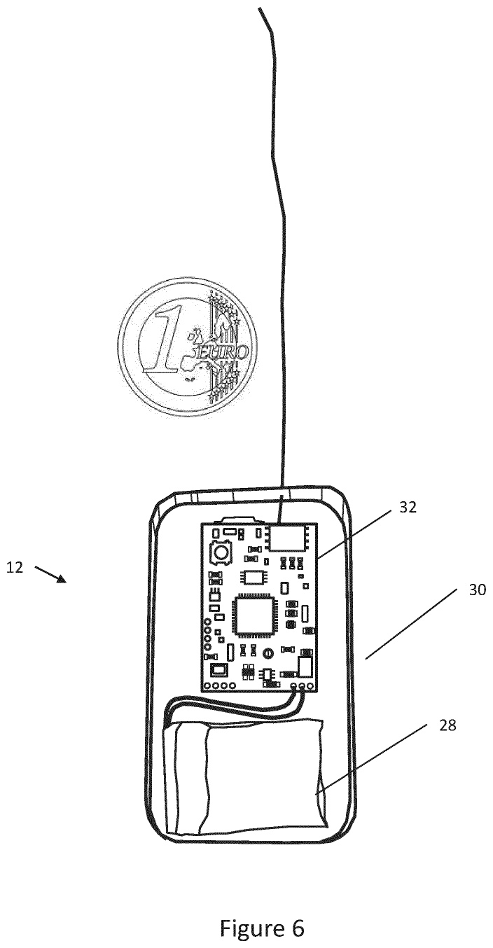

FIG. 6 shows a rectangular and slimmer embodiment of the sensor tag 12, suitable for direct attachment to the cow's tail 16 with adhesive and/or Velcro strips. For 868 MHz LoRa RF transmission, an 8.6 cm quarter-wavelength wire antenna is shown. Alternatively this antenna could be a loop coil on the PCB, or a helical coil structure in the layers of the PCB. The skilled reader will understand that other sub-gigahertz frequencies may be used and the wire antenna adjusted as appropriate to give higher antenna efficiency and ensure data is not lost during transmission.

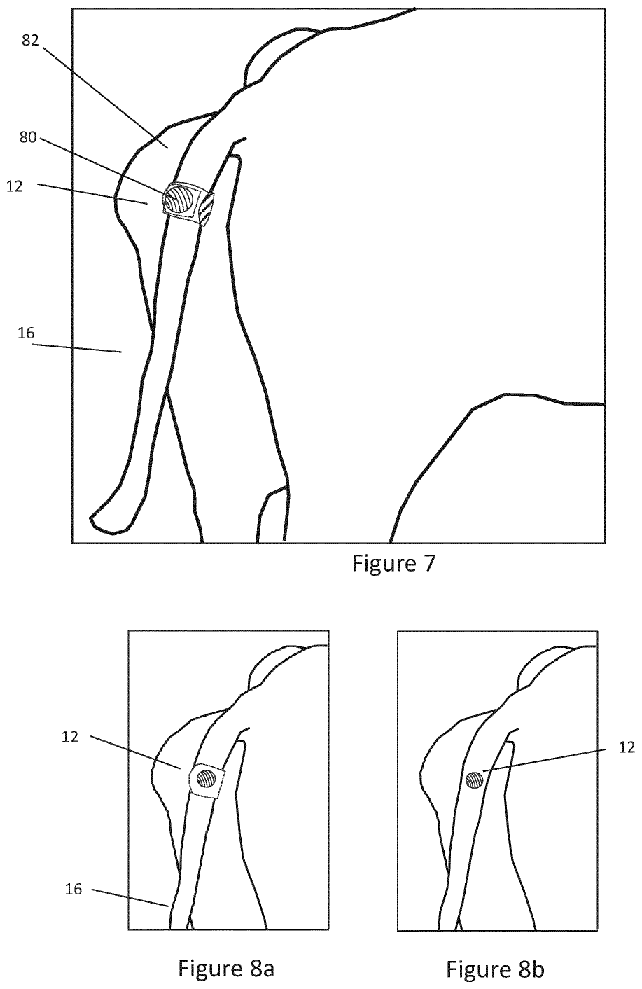

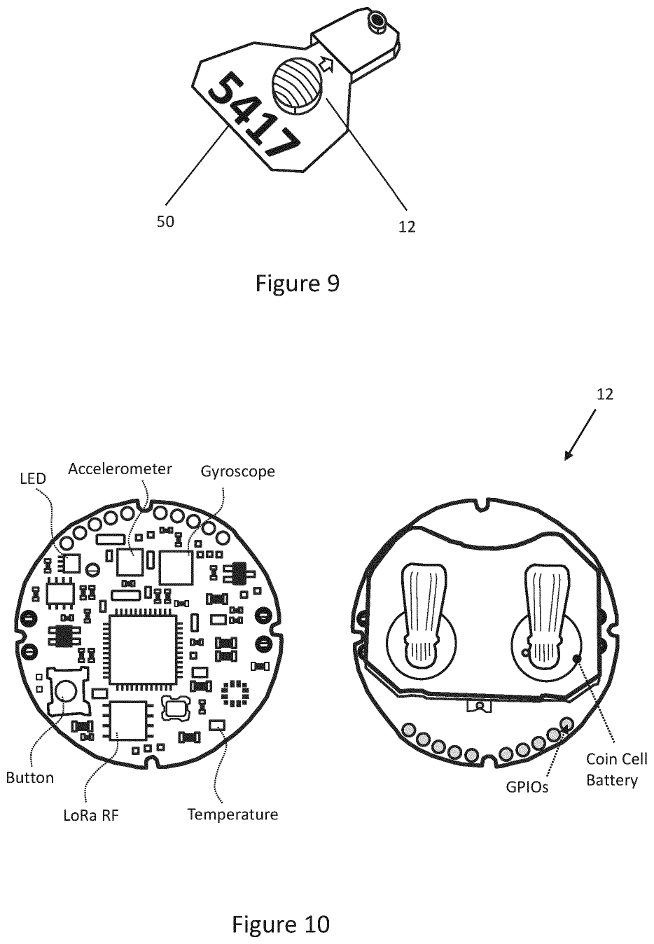

The sensor 12 may be secured to the tail 16 with an adhesive or with a medical grade crepe elastane bandage using Velcro. FIG. 7 shows the sensor tag 12 secured to the tail 16 by a fabric strap secured by a Velcro attachment. The fabric strap 80 comprises a pocket within which the sensor tag 12 may be received. The farmer may apply a bovine adhesive, for example a glob of Kamar tag-glue, on the inside of the fabric strap 80 shown in FIG. 7 prior to securing the fabric strap 80 with Velcro to the tail 16. This facilitates simple and rapid attachment, in a few seconds, for example less than 10 seconds. The adhesive or glue stops the sensor tag 12 rotating or sliding relative to the tail 16, and the soft fabric strap 80 does not hurt the cow or cause soreness to the tail as is the case with previous solutions. Furthermore, locating the sensor tag 12 within a hidden pocket reduces the chance of the tag 12 becoming caught and dislodged from the cow's tail 16.

FIG. 8a shows the sensor tag of FIG. 6 with a glob of adhesive placed on the cow's tail and wrapped with a conformal fabric bandage material, elastane for example. This is beneficial as the elastane bandage may further improve the attachment method of securing the sensor tag 12 to the tail of the cow 10. In this embodiment the tag 12 may be secured to the tail 16 by the adhesive and/or an attachment element such as Velcro. The skilled reader will appreciate that soft breathable fabric materials other than an elastane bandage may be wrapped around the tail of the cow to secure the tag 12. The elastane bandage is beneficial due to its breathable nature and comfort to the cow.

FIG. 8b shows a 5 gram 15 mm diameter embodiment, glued directly to the outer tail hairs, with no bandage or fabric. This is similar to a piece of dirt stuck to the tail, almost imperceptible to the cow. It can only be removed by cutting off or pulling off (or molting) of the tail-hairs.

These attachment methods, and the lightweight nature of the sensor tag, are advantageous as they cause minimal discomfort to the cow. This eliminates the well-known issues of heavier sensors which the cow tries to knock off due to annoyance, or which causes sores or swelling of her tail due to the tight clamping required to hold them in position.

Cattle are often fitted with electronic ear tags. The ear-tags comprise an RFID chip that contains information relating to the animal the electronic ear tag is fitted to. For example, the RFID may contain a unique animal identification number that contains information about the animals. In an embodiment, the sensor tag 12 may comprise a near field communication (NFC) module that is configured to communicate with the electronic ear tag. In this embodiment the sensor tag 12 may be held in the vicinity of the ear tag prior to being secured to the cow 10, for example within 300 mm of the ear tag, or within 30 mm of a HF ear tag. The sensor tag 12 may communicate with the electronic ear tag such that the unique animal identification number is read and stored by the sensor tag 12. The sensor tag 12 is configured to determine data indicative of the breed and calving history of the animal, based on the unique animal identification number, from its pre-stored memory, or by requesting the previous history from a cloud database via the gateway 14. It can then adjust and tailor its learning algorithm coefficients accordingly to the oestrus and calving behaviour of each individual cow 10. The cloud database may additionally contain data from other cows in the herd. This enables analysis of individual cow movements versus herd-level cow movements, resulting in increased accuracy of oestrus determination.

With non-electronic ID ear-tags, the farmer may match the sensor tag to cow by photographing the ear-tag number with his phone, while holding the tag within 300 mm of the phone. The phone digitizes the cow's ID number, and based on signal strength, it pairs with the nearby tag, not other tags which may be in the vicinity. The tag then reads and stores the ID number, as the farmer then attaches it to the said cow.

Mounting an embodiment of the sensor tag 12 to the ear or neck of the cow (12c, 12d FIG. 1) is advantageous as it enables the tag to monitor the known changes in traits and behaviours during oestrus, such as movements of the cow's head, e.g. extra movement & butting, as well as extra walking, changes in grazing patterns, standing for mounting, less feeding, more restlessness and lying/standing bouts, etc. These behaviour and movement changes are particularly beneficial for determining when the cow 10 is in heat and ready for insemination.

FIG. 10 shows a perspective view of an electronic ear tag 50 prior to being secured to the ear of the cow 10. The sensor tag 12 may be secured to the electronic ear tag 50. This is beneficial as it is non-intrusive to the cow and may be easily clipped to the ear tag 50 by the farmer. The tag may also be secured directly to the ear of the cow 10 by a tag-attach gun or the like. FIG. 11 shows the sensor tag 12 as shown in FIG. 10 with the outer casing removed. It has a coin-cell battery e.g. CR2032 250 mAh, to enable several years of operation, employing low-duty-cycling and kB/s low data-rates. This embodiment is not rechargeable, and when closed, is completely sealed.

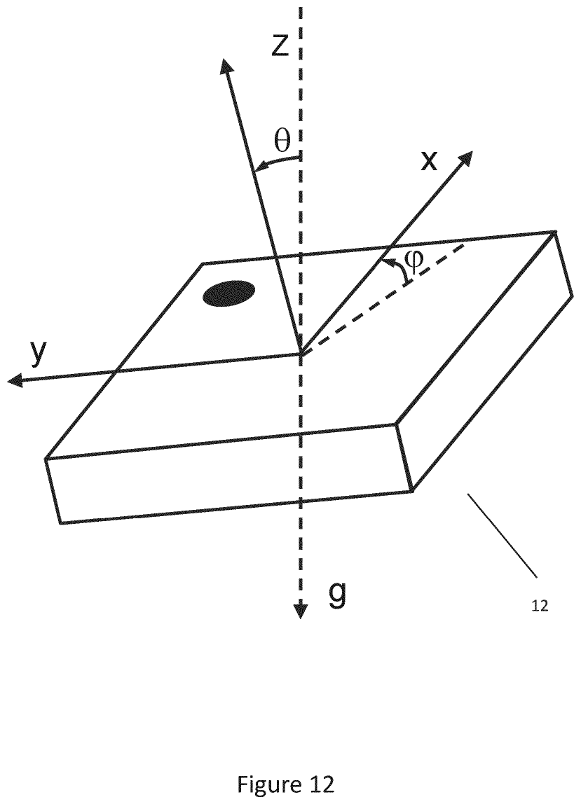

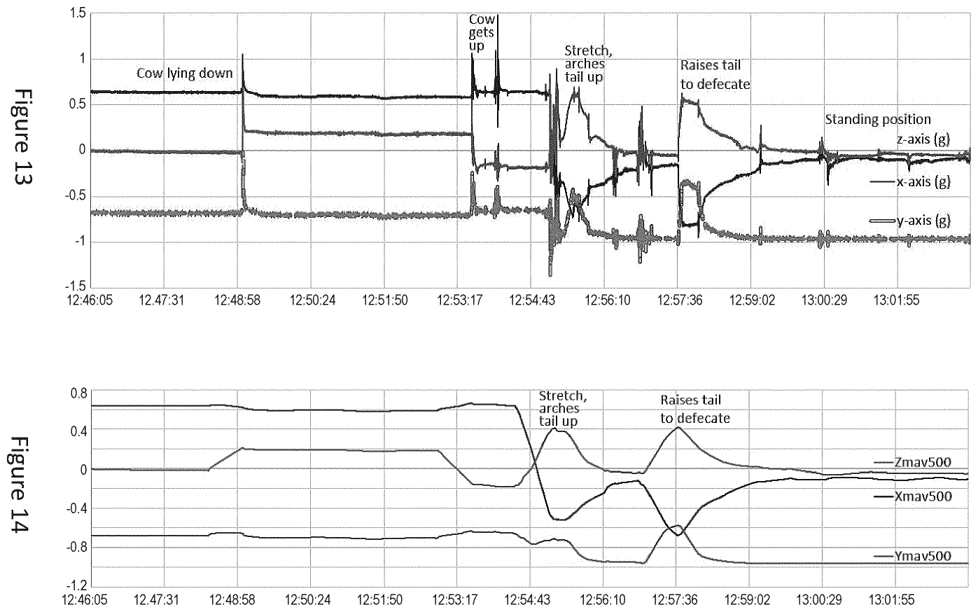

Turning to FIG. 12, an embodiment of the sensor tag 12 may also be secured to the leg of the cow 10. Securing the sensor tag 12 to the leg of the cow 10 allows the sensor tag 12 to monitor the cow's walking and movement patterns. This embodiment is also suitable for detecting when the cow 10 is in heat and ready for insemination. The sensor tag 12 may be secured to the leg of the cow 10 by a strap 80 as shown in FIG. 11.

The sensor tag 12 is light enough to be easily secured to the cow's leg, ear or tail 16 such that it is almost imperceptible to the cow 10. Conversely, the neck-mounted embodiment can be heavier, facilitating the use of a larger battery and integration of a GPS location sensor and a GSM wireless module. Thus the neck-mounted sensor can also function as the gateway unit 14 in this embodiment, communicating locally with the lightweight sensor tag 12 or tags on the cows leg 12b, ear 12c, or tail 12a, for example by Bluetooth Low Energy (BLE) pairing, and communicating with the farmer or cloud database by GSM. It can thus send the farmer an alert for oestrus and/or calving, together with the cows ID number and exact location. This is particularly beneficial for suckler animals who may be roaming fields for extended periods, out of range of farm-shed based antenna systems.

FIG. 11 shows a hardware diagram of a sensor tag 12 according to another embodiment. As shown in FIG. 14, the sensor 12 may comprise a wireless induction charging unit. The wireless induction unit comprises a flexible induction coil mounted or printed on the inside cover of the tag 12. This is beneficial as the outer casing of the sensor tag 12 may be completely sealed to protect the sensor tag 12 from the external environment and allowing the farmer to easily clean and wash the sensor 12 after use. The wireless charging unit removes the requirement for a charging port in the external casing of the sensor tag 12 thereby improving the quality of the seal of the external casing. The external casing may be wrapped in a water-proof material, such as a plastic film, to further improve the seal of the outer casing.

In another embodiment of the sensor tag when mounted on the tail, the antenna and wireless charging coils assist detection of oestrus by detuning slightly when the cow is being mounted by another animal. This is because the large mass of another animal in close proximity to tag changes the stray capacitance and electric field, which the RF receiver is programmed to detect. Or alternatively the capacitance variation may be measured directly, for example by a charge-balancing second order sigma delta converter in the tag. This resolves 1 femtoFarad to 10 femtoFarad capacitance variation for another animal in 15 to 150 mm proximity of the tag. In combination with the other X,Y,Z movement sensors, the sensor tag can therefore make a much more accurate estimation of “standing” or “not standing” oestrus/‘in-heat’ status of the cow. Normal animal proximities, during feeding at troughs for example, which could cause false oestrus alarms, can be ruled out by the machine-learning algorithm which is aware of the cows behaviour and movement patterns over hours, days, or weeks, and by Bayes and Kalman recursive filter analysis of these patterns. This reduces or eliminates such false alarms

In more detail, the algorithm calculates an ‘activity index’ based on the cow's movements, number of steps, lying/standing bouts, and angle and frequency of tail movements, also combining tail proximity sensor data. It also calculates a ‘probability index’ of reaching a correct detection conclusion based on pattern-matching classification and recursive sample analysis. The movement sensors 22 are configured to measure the X, Y and Z accelerations between, for example, one and ten times per second, and calculate the gravity vectors, as per the following equations with reference to FIG. 12:

acceleration_x=1 g*sin θ*cos ψ

acceleration_y=−1 g*sin θ*sin ψ

acceleration_z=1 g*cos θ

Tracking the X, Y and Z gravity vectors identifies the cow's position status, for example, is the cow 10 standing up or lying down. Furthermore, when the cow 10 is lying down the control module 20 is configured to determine if the cow 10 is lying on its stomach or either of its left or right sides. When the control module 20 determines the cow 10 is in a lying position it reduces the rate at which it measures the accelerations to, for example, once per second to conserve the battery of the sensor tag 12 even further. Furthermore, when the control module 20 determines that the cow 10 is active the rate at which it measures the accelerations may be increased, for example 10 or 20 times per second. In a broad sense, the control module 20 is configured to vary the sampling rate or duty cycle in dependence on the activity of the cow 10.

The control module 20 is further configured to determine linear accelerations. Linear accelerations are a derivative of cow's position status (standing, lying), and provide information indicative of the movement, walking and pacing of the cow 10. This may be measured when the sensor tag 12 is mounted in either the ear tag or to the tail of the cow 10. Furthermore, when the sensor tag 12 is mounted to the tail of the cow 10 the sensor tag 12 may track the movement of the tail 16. For example, the sensor tag 12 is configured to track the angle of the tail and distinguish for example contractions during labour from urination, defecation, and swishes of the tail. This is beneficial as indicative of the cow calving while minimising false positive alerts.

The movement sensor 22 may comprise an accelerometer and one or more of a gyroscope and a magnetometer. In embodiments that comprise a magnetometer and/or a gyroscope in addition to the accelerometer, the control module may activate the gyroscope to cross reference data points to assist in the control module determining parameters of the cow such as determining when the cow is in heat or when the cow is calving. The gyroscope and magnetometer consume more power than the accelerometer and as such the control module activates these movement sensors sparingly to cross reference data from the accelerometer in establishing its activity and probability indexes. Typically, the gyroscope and magnetometer consume up to approximately 1 mA of current compared to 130 pA for the accelerometers. Thus for a cow calving in a pen, where direction and orientation are not important for birthing detection, only the accelerometers may be required in reaching the birthing alert.

The gyroscope and magnetometer provide further advantages when the cow is calving in a field on a farm. For example, the magnetometer may determine the direction in which the cow is facing. Advantageously, this data may be used in conjunction with the number of steps the cow is determined to have taken to notify the farmer of an approximate location of the cow 10. Cow's often move to a secluded location, away from the herd, during a period of calving making them difficult to locate by the farmer. As such the notification transmitted to the farmer's mobile communication device may include an alert of an expected calving time and an approximate location of the cow 10.

The skilled reader will appreciate that the present invention may be implemented with a movement sensor 22 that comprises one or more of an accelerometer, a gyroscope or a magnetometer. For example, the movement sensor 22 may comprise only an accelerometer configured to determine movements of the cow or the movement sensor 22 may comprise a plurality of different sensors configured to operate in conjunction with each other to track and verify movements of the cow and the cow's tail.

The algorithm implemented on the control module is configured to extract known positions (standing, lying-left, lying-right) from the movement sensor data. It calculates the ratio of cow standing time (mins/hr) versus time lying down (mins/hr), and the number of Lying Bouts. A high-pass filter may be used on the data to extract walking, number of steps, and movement patterns.

For calving detection, the algorithm then uses these calculations and the movement sensor data inputs to establish an activity index, Alx, and a probability index Plx to maximise the likelihood of reaching a correct birth-alert decision in a narrow time-frame of 1 to 3 hours before birth. The algorithm does this by training itself to adapt and learn movement and data patterns that may be unique to the cow 10 that the sensor tag 12 is fitted to, as well as adapting based on her breed, previous birthing history, primiparous vs multiparous, etc. This improves the accuracy of the pattern matching step as the algorithm may learn movement patterns that are typical of the cow when she is not in heat or calving and compare the known patterns with a stored pattern in conjunction with the determined activity index and probability index.

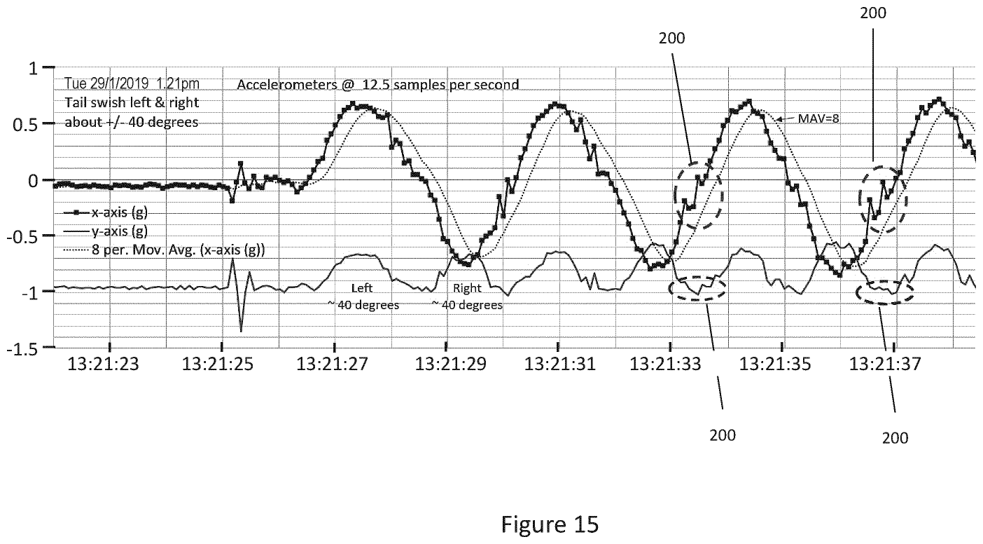

Furthermore, the algorithm may employ recursive Kalman and Bayes filtering techniques to deal with predictable and unpredictable noise, uncertainties, and errors in the calving or oestrus measurements. Some examples are as follows:
FIG. 13 is a 20-minute snapshot of movement sensor data of the cow lying down, getting up, then her tail raises twice—a stretch, followed by a defecation. FIG. 14 is the moving-average-filtered version of this. The two tail-raises are each about 30-seconds duration, and about 3-minutes apart. These are almost identical to stage-2 labour calving contractions. The algorithm distinguishes these from real contractions by recursively going back to the movement and position data gathered and stored in the previous hours in the tag, and uses Bayes probability scoring to confirm labour has not started. This estimates the probability density function recursively over time using the current incoming measurements and the stage-1/stage-2 labour/parturition model stored in the tag's memory, and uses this to adjust the Probability Index (Plx) if and as required.
In FIG. 15 the circled areas 200 are an example of gimbal uncertainty, where errors of up to 0.15 g occur in X_accel as it passes through zero, due to Z also being close to zero and Y_accel vector becoming parallel to gravity, i.e. the point of gimbal-lock uncertainty, in which the sin/cos equations become slightly unstable. A low-pass filter (MAV=8) smooths out the noise in this case. But if the cow is fairly static in such a gimbal-lock position, then a moving-average-filter will not fully eliminate this. Kalman uncertainty estimation may produce more accurate estimates of actual position in this situation.

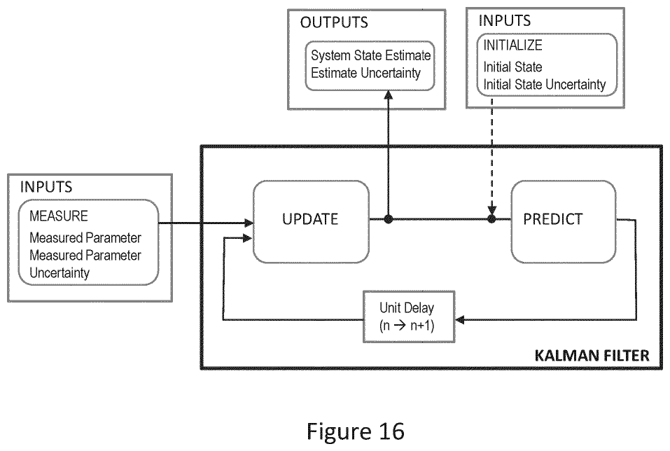

FIG. 16 shows a block diagram of a Kalman filter suitable for use with embodiments of the present invention. An example of the Kalman Gain Equation is shown below:

$${K}_{n} = \frac{Uncertainty in Measurement}{Uncertainty in Estimate + Uncertainty in Measurement} = \frac{{p}_{n , n - 1}}{{p}_{n , n - 1} + {r}_{n}}$$

In the above Kalman Gain equation p_{n,n-1} is the extrapolated estimate uncertainty and rₙ is the measurement uncertainty.

Furthermore, the algorithm may implement a Bayes filter to deal with sudden and unpredictable changes and increases of the cow's movement patterns. For example, in response to a sudden incursion by a cat or dog that may startle the cow or, for example, at feeding time where the cow may become excited and move in an unpredictable and erratic manner. Situations like the above often resulted in false-positive notifications for the farmer when using systems typical of the prior art.

The memory of the sensor tag 12 is configured to store movement data gathered by the movement sensors 22. The memory module typically may store ten to one hundred or more days of movement data gathered by the motion sensors 22.

For oestrus detection, the algorithm may similarly generate a mathematical function of the cows movements, walking and lying patterns, and neck and head movement patterns. It may similarly employ Bayes and Kalman recursive filtering methods to distinguish normal cow movement and proximity data from ‘in-heat’ oestrus movement patterns.

In another embodiment the memory module may be located on a remote PC or cloud computing device. In this embodiment the tag 12 relays data to the gateway unit 15 which may then forward the data to the mobile communication device 15. This is advantageous as movement and oestrus or calving data of each cow 10 may be stored on a remote memory module and accessed the following year at oestrus or calving time. This would allow the control module to recall movement patterns of the cow 10 from previous calving and oestrus cycles and to update the algorithm accordingly to tailor the algorithm to each cow 10. The data may be retrieved from the remote memory module upon holding the sensor tag 12 near to the electronic ear tag of the cow 10. The NFC would communicate with the electronic tag to identify the cow 10 to which the tag 12 is being secured, at which point calving data relevant for that cow would be transmitted to the sensor tag 12 from the mobile communication device 15.

Because the tag is so light, attached to the cow's tail 16 with no soreness or side-effects, it can be put on the tail at least one or two weeks before the expected calving date. Unlike all other sensors, longer time on the tail is advantageous in allowing better ‘learning’ by the machine learning algorithm of the cow's movement and behaviour patterns. In the event of sudden changes in the cow's activity, the algorithm can look-back′ over the previous hours and days of data to help in deciding whether or not to issue a birth alert. For example, a cow's sudden excitement and activity level during feeding and defecating (which causes false alarms in other sensors) can quickly be adjudicated simply by looking back through memory at her movement history in the preceding hours and days, and ruling out a birth alert if there are no signs of contractions.

When fitting the tag 12 to the cow 10 the farmer is required to input parameters indicative of the cow the tag is to be fitted to prior to fitting the tag 12 to the cow. The farmer may do this by holding the tag 12 in the proximity of the cow's electronic ear tag 80 such that the tag recognises the cow's unique ID number and can automatically retrieve data parameters relevant to the cow or he may manually input the data on the mobile communication device 15 prior to securing the tag to the cow 10. Examples of the relevant data parameters include but are not limited to: the cow's ID number, the breed, her calving history, the number of calves she has previously had, an expected due date and whether she has already started labour and an approximate feeding time. Other data parameters relevant to the cows calving movements may be added by the farmer as appropriate.

The data parameters may be entered in the tag 12 by the farmer via a series of Q & A text messages between his phone and the tag or by using the NFC feature of the tag 12 by holding the sensor tag 12 next to her (electronic) ear-tag. When using the NFC feature the calving tag reads her ID number (via the NFC RF chip), and then can download all the cow's relevant details: her breed; her previous birthing history etc. The sensor tag 12 can then tune and adapt the algorithm to suit the cow 10. For example, for an Angus cow or a Shorthorn cow the sensor tag 12 will identify that they are ‘early’ calvers—compared to Limousin or Charolais cows, who often go 2 to 4 weeks beyond due date. Similarly, if the breed is a Belgian Blue, the tag 12 will know that they nearly always need a caesarean birth, where all these factors and coefficients become even more important and the sensor tag 12 may monitor the cow more closely for any signs of distress and difficulty at which point a notification will be sent to the farmer.

Typically, the farmer will secure the sensor tag 12 to the tail 16 of the cow 10 for determining when a cow is calving although the skilled reader will understand that the sensor tag 12 may also be secured to any one of the ear, the leg or the tail of the cow depending on whether the farmer wants to detect when the cow is calving or when the cow is in heat.

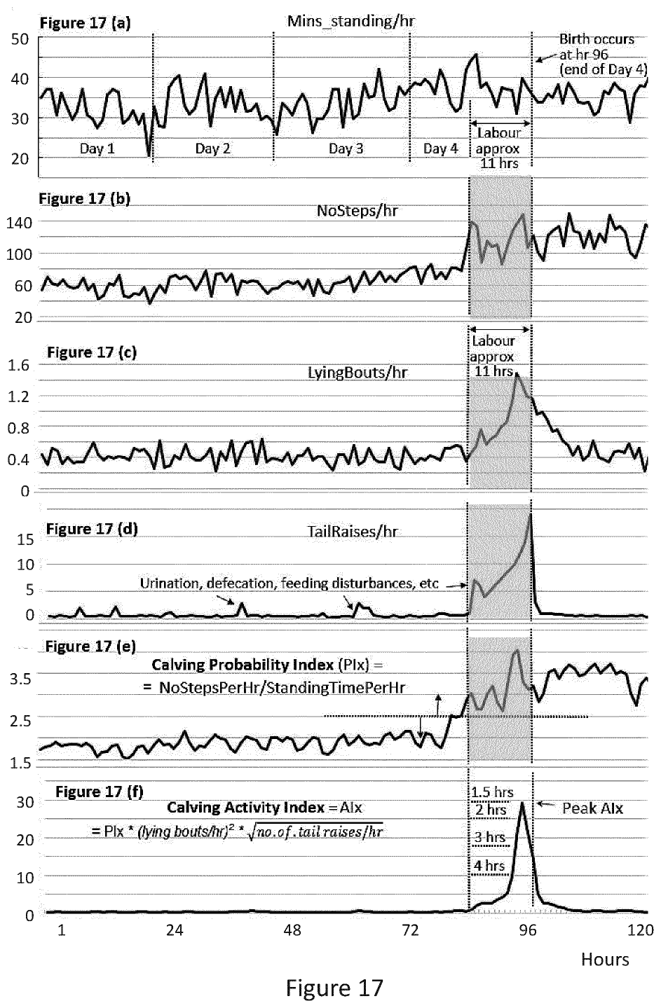

FIG. 17 shows graphs of sensor tag data and movement patterns gathered from a sensor tag 12 on a Holstein Friesian cow over five days, with calving occurring on the 4^{th} day (hour 96). The labour period of approximately 11 hours and is shown shaded:

The movement data and resulting calculations are shown as follows:
FIG. 27(a) is the cow's standing time (mins/hr);
FIG. 27(b) is the cow's number of steps per hour;
FIG. 27(c) is the cow's number of Lying Bouts per hour;
FIG. 27(d) is the cow's number of Tail-Raises per hour;
FIG. 27(e) is a Calving Probability Index, calculated in this particular embodiment as

$$Calving Probability Index Plx = \frac{number of steps / hour}{standing time / hr}$$

If Plx>2.5, there is a high probability the cow as started labour;
If Plx <2.5, the cow is very unlikely to be in labour.
FIG. 27(f) is a Calving Activity Index, calculated by the sensor tag in this embodiment as

Calving Activity Index Alx=Plx*(lying bouts/hr)²*\sqrt{no.of.tail raises/hr}

Alx is a very good predictor of calving, with the peak Alx being 1.5 hours before birth. The dotted lines show the algorithm adjusting to other prediction thresholds (2 to 4 hours) depending on cow breed, calving history, and other factors as previously described herein. Beneficially, the sensor tag 12 may monitor both the steps and movement of the cow as well as the movement of the cow's tail 16. This provides a more reliable and accurate prediction of when the cow is about to calve. For example, the sensor tag 12 may notify the farmer that the cow is about to calve 1.5 hours prior to calving.

The sensor tag 12 is configured to provide a calving notification to a mobile communication device when the probability index and/or the activity index of the cow 10 exceed a threshold value. The algorithm on the control module may vary the threshold at which the calving notification is generated in dependence on the cow 10. Furthermore, the control module 20 may learn an activity index pattern or movement pattern over a period of time prior to calving such that the algorithm may learn a typical activity index or movement pattern of the cow 10. The sensor tag 12 may then detect a change in the movement pattern or activity index and probability index that is indicative of the cow calving.

In an embodiment the farmer may adjust the time at which a notification is provided to the mobile communication device. For example, the farmer may indicate that they would like to receive a notification 1 hour prior to the expected calving time or they may indicate that they would like to be notified further in advance in which case the notification may be provide, for example, 4 hours prior to calving.

It will be appreciated that various changes and modifications can be made to the present invention without departing from the scope of the present application.

1. A method of determining when a pregnant cow is about to calve, the method comprising:
monitoring movement of the cow using a motion sensor attached to the cow;
determining a mathematical function of a movement pattern of the cow based on the monitored movement of the cow over a period of time wherein the mathematical function is a calving activity index; and
determining that the cow is about to calve when the calving activity index exceeds a threshold value; wherein the threshold value is adjusted up or down by a probability index indicative of the probability the cow has started labour.

2. The method as claimed in claim 1, wherein the probability index is determined by dividing the number of steps the cow has taken within a time period by the time the cow spent standing in said time period.

3. A method as claimed in claim 1 or claim 2, wherein the threshold value is 10.

4. A method as claimed in claim 1 or claim 2, wherein the threshold value is 20.

5. The method in as claimed in claim 1, wherein the calving activity index is calculated by multiplying the probability index by: (lying bouts/hr)²*\sqrt{no.of.tail raises/hr}.

6. The method as claimed in claim 1, wherein the method comprises generating an alert that the cow is about to calve.

7. A method as claimed in any preceding claim, wherein the threshold value is adjusted in dependence on the breed of the cow the motion sensor is attached to.

8. The method as claimed in claim 1, wherein the threshold value is adjusted in dependence on a calving history of the cow.

9. The method as claimed in claim 1, wherein the method comprises scanning an electronic ID tag of the cow and adjusting the threshold value in dependence on the scanned electronic ID tag.

10. The method as claimed in claim 1, wherein the method comprises scanning a non-electronic ID tag of the cow and adjusting the threshold in dependence on the scanned non-electronic ID tag.

11. The method as claimed in claim 1, wherein the method comprises adjusting a duty cycle of the motion sensor in dependence on the movement pattern of the cow.

12. The method as claimed in claim 11, wherein the method comprises reducing the duty cycle of the sensor tag when the cow is lying down.

13. The method as claimed in claim 11, wherein the method comprises increasing the duty cycle of the sensor tag when the cow is standing up.

14.-26. (canceled)

27. A self-powered motion sensor tag that is attachable to the tail of a cow to determine when the cow is about to calve by reference to a mathematical function of the cow's movements, wherein the tag is configured to emit a wireless signal that is indicative of the cow calving and comprises:
an adhesive for attaching the tag to the tail of the cow; and
a housing containing:
at least one three-axis motion sensor for determining the cow's movements;
a controller that is responsive to the motion sensor to generate said mathematical function and said signal, wherein the mathematical function is a calving activity index indicative of the probability the cow has started labour and
a wireless communication module and an antenna for emitting said signal wirelessly, wherein
the tag weighs less than 20 grams and is less than 30 mm diameter.

28. The tag of claim 27, wherein the tag is less than 10 grams and less than 25 mm diameter.

29. The tag of claim 27, wherein the tag is attached to the outer hairs of the tail.

30. The tag of claim 29, wherein the tag is further secured on the tail hairs by a wrap-around breathable fabric.

31. The tag of any one of claim 29 or 30 wherein the sensor tag can only be removed by cutting or pulling out the tail hairs, or by moulting of the hairs.

32. The tag of claim 27, wherein the calving activity index is calculated by a processor in the controller as a calving probability index multiplied by (lying bouts/hr)²*\sqrt{no.of.tail raises/hr}.

33. The tag of claim 32, wherein the calving probability index is calculated by dividing the number of steps the cow has taken within a time period by the time the cow spent standing in said time period.

34. The tag of any one of claims 27 to 33, wherein the calving activity index is compared to a threshold value.

35. The tag of claim 27, wherein the controller comprises a Bayes filter and/or a Kalman filter.

36. The tag of claim 35, wherein the filter is configured to filter movement data generated by the movement sensor and to vary the calving activity index up or down in dependence on the filtered movement data.

37. The tag of claim 27, wherein the range of the tag's emitted wireless signal is a maximum of 50 metres.

38. The tag of claim 27, wherein the range of the tag's emitted wireless signal is a maximum of 20 metres.

39.-76. (canceled)
